(19) United States
(12) Patent Application Publication
(10) Pub. No.: US 2017/0055891 A1
(43) Pub. Date: Mar. 2, 2017

(54) METHOD, DEVICE AND SYSTEM FOR NON-INVASIVE MEASUREMENT OF BLOOD GLUCOSE CONTENT

(71) Applicant: Leila Chaychi, Redwood City, CA (US)

(72) Inventor: Leila Chaychi, Redwood City, CA (US)

(21) Appl. No.: 14/842,832

(22) Filed: Sep. 2, 2015

(51) Int. Cl. A61B 5/145; A61B 5/00; A61B 5/1455

(52) U.S. Cl. CPC A61B 5/14532; A61B 5/14551; A61B 5/681; A61B 5/6831

(57) ABSTRACT

An accurate and real-time architecture that uses Near Infra-red (NIR) and mid infra-red (IR) thermal energy to determine blood glucose levels in a patient's body member based on NIR and IR transmittance and reflecting back while in contact with the body member.

### TECHNICAL FIELD

The present disclosure relates to the field of blood glucose management and, more particularly, to methods, devices and systems for non-invasive measurement of blood glucose content at a site having a vasculature.

### BACKGROUND

Diabetes mellitus (DM), or commonly referred to as diabetes, is a group of metabolic illnesses that result in elevated blood glucose content. There are three main types of diabetes: (1) Type 1 caused by the inability of the pancreas to secrete sufficient insulin, a hormone that regulates the metabolism of carbohydrates and sugars; (2) Type 2 due to insulin resistance; and (3) Gestational when a pregnant woman with no prior history of diabetes develops high blood sugar level during pregnancy.

Patients are diagnosed with different types of diabetes every day. Today, 387 million individuals have diabetes around the globe, and it is estimated that by 2035, this number will rise to 592 million patients according to the International Diabetes Federation. Diabetes Atlas, Sixth Edition. International Diabetes Federation, 2014. Once diagnosed with diabetes, a whole set of outcomes arises such as the substantial economic burden of paying for care and treatment, which often involves monitoring and managing the blood sugar level by insulin injections. These costs need to be borne by patients, health care systems and governments. In 2012, the total estimated direct and indirect medical costs associated with diagnosis and treatment of diabetes in the U.S. alone was of $245 billion. This represents a significant increase of 41% since 2007 according to the American Diabetes Association.

Various insulin monitoring methods and instruments have been proposed in order to assist patients with metering their blood glucose level in order to maintain it at an optimal range. However, the most popular existing insulin measurement techniques involve drawing a blood sample from the patient's arteries and analyzing the blood sample in the laboratory, or obtaining a capillary blood sample through a small pinprick with a lancing device and placing the blood sample on a test strip for analysis by as patient-owned bG meters or continuous glucose monitors. These existing techniques suffer from a number of drawbacks. They are invasive, pose risk of infection, costly and cause discomfort for the patients. Moreover, the existing patient-owned glucose detection devices are prone to inaccuracies and provide values with a wide deviation, which may fall erroneously within the acceptable range for a patient.

Although some non-invasive ways to measure the blood glucose such as transdermal, ultrasonic, electromagnetic, and thermal measurement also exist, they are not as effective and accurate as the invasive ones. Many of the current non-invasive methods use technologies use incident radiation in order to reach the blood by penetrating through the tissue and measuring the reflected radiation to extrapolate the blood glucose content. However, these methods suffer from lack of accuracy as there is often only one parameter that is being measured. This form of measurement does not assure enough precision to determine precisely the blood glucose levels. Although some of the non-invasive proposed solutions seek to improve accuracy by using different sensors, these solutions are not very easy to use and require the patient to align various sensors on a body part such as the earlobe. Unless the patient has immediate access to a mirror, the precision of such a performance could be significantly compromised by misalignment of the various sensors.

Accordingly, there is a need for a method, device, and system for measuring blood glucose content that seeks to address the shortcomings of the existing glucose metering devices.

### SUMMARY

In one example embodiment, a user device for non-invasive measurement of blood glucose content in a site having a vasculature is disclosed. The user device comprises a display and a first detector configured to detect red, infra-red (IR), and near infra-red (NIR) signals reflected from glucose contained in the blood at the site. The user device further includes a communication interface coupled to the first detector, at least one processor in communication with the communication interface and configured to: detect the concentration of glucose contained in the blood at the site based on IR signal detected by the first detector; and display a number representative of the concentration on the display. In some examples, the user device further comprises at least one detector configured to detect the oxygen concentration contained in blood.

A method for non-invasive measurement of blood glucose content in a site having a vasculature is provided. The method includes the steps of detecting the concentration of glucose contained in the blood based on the radiation (such as red, IR or NIR) radiation detected at the site, and displaying a number representative of the concentration on a display. The method can also include a further step of detecting the concentration of oxygen contained in the blood.

In another example, a kit for non-invasive measurement of concentration of glucose contained in the blood in a site having a vasculature is disclosed. The kit has a first detector configured to detect red, IR, and NIR signals reflected from glucose contained in blood. The kit also includes a user device having a communication interface coupled to the first detector, at least one processor in communication with the communication interface and configured to: detect the concentration of glucose contained in the blood at the site based on IR signal detected by the first detector, and display a number representative of the concentration on the display.

### BRIEF DESCRIPTION OF THE DRAWINGS

The accompanying drawings, which are incorporated in and constitute part of this disclosure, together with the description, illustrate and serve to explain the principles of various example embodiments herein.

FIGS. 1A-1B are diagrams of example user devices in which various implementations described herein may be practiced.

FIG. 2 is a diagram of a user device for implementing embodiments consistent with the present disclosure.

FIG. 3 is a graph showing the reference and predicted correlation of blood glucose concentration.

FIG. 4 is a flow chart showing a method for measuring blood glucose content in accordance with the present disclosure.

### DETAILED DESCRIPTION OF EXAMPLE EMBODIMENTS

Reference will now be made in detail to the example embodiments implemented according to the present disclosure, the examples of which are illustrated in the accompanying drawings. Wherever possible, the same reference numbers will be used throughout the drawings to refer to the same or like parts.

Self-monitoring blood glucose metering devices are particularly desirable for patients with symptoms or with a history of abnormally high or low blood glucose levels. The possibility of self-monitoring the amount of glucose in the blood has an effective impact on the outcome of the diabetes treatment. This method of self-monitoring of the glucose in the blood helps patients take control through automatic feedback and adjust their lifestyles before serious long-term damage arises. Moreover, the self-monitoring devices enable diabetic patients to administer appropriate insulin in the convenience of their homes or workplace, without the requirement of clinical-grade diagnostic equipment or analysis. This immediate access to vital information also helps the medical team with up-to-date and particularized data on the patient's blood glucose levels.

However, the current self-monitoring blood glucose devices commonly used by diabetes patients are invasive and have proven to be expensive, painful and complicated to use. They are also not practical since they require the transportation of an external device and the regular extraction of blood from the patient which leads to discomfort and even infections. All of these elements are important factors that contribute to the reduction of the use of self-monitoring blood glucose. This is a major problem considering how important the regular evaluation of the blood glucose is important for the health and the long-term cost reduction for diabetes patients. This is why it is particularly advantages to find a painless, inexpensive, user friendly and practical way to measure the blood glucose. Such a device would come to would further encourage the diabetes patients to test their blood glucose more frequently and avoid hypoglycemia or hyperglycemia.

The solution herein addresses a pressing need for less expensive, painless and easy to use method of measuring blood glucose level that is non-invasive. An accurate and real-time architecture for blood glucose measurement is proposed. Since electronic devices such as mobile devices (for example smartphones, wearable devices such as wearable watches or wearable fitness devices, netbooks, tablets, gaming consoles or the like) are becoming ubiquitous, the solution herein can be implemented on an electronic device that is handheld and/or portable to detect through a non-invasive process the blood glucose levels. In this manner, the patient will not have the burden to carry a separate self-monitoring blood glucose device around and will be able to monitor the glucose level at any time. Moreover, additional features of the mobile device, such as advance data processing, web-enabled wireless communications, and enhanced color display or audio interfaces, in order to provide the patient or medical staff with improved presentation, transfer, or notification of data concerning the patient's insulin level.

As explained in further detail, an app being executed on the user device can collect the blood glucose level measurements from a patient's member such as a thumb through Near-Infrared (NIR) thermal energy. By placing the thumb proximate to a NIR sensor coupled with the user device, the NIR transmittance would be paired with various body parameters such as tissue thickness, blood oxygen saturation and a linear regression-analysis based calibration system.

Because there is a need for accurately measuring glucose level in diabetes patients, the solution herein seeks to measure the blood glucose level by the use of three non-invasive protocols, namely, ultra sound, electromagnetic, and thermal technologies that do not require the often painful prick of a finger or the draw of blood.

Embodiments of the present disclosure provide methods, user devices and systems for non-invasive measurement of blood glucose content.

The embodiments herein also include computer-implemented methods, tangible non-transitory computer-readable mediums, and systems. The computer-implemented methods can be executed, for example, by at least one processor that receives instructions from a non-transitory computer-readable storage medium. Similarly, systems and devices consistent with the present disclosure can include at least one processor and memory, and the memory can be a non-transitory computer-readable storage medium. As used herein, a non-transitory computer-readable storage medium refers to any type of physical memory on which information or data readable by at least one processor can be stored. Examples include random access memory (RAM), read-only memory (ROM), volatile or nonvolatile memory, hard drives, CD ROMs, DVDs, flash drives, memory sticks, memory disks, and any other known physical storage medium. Singular terms, such as “memory” and “computer-readable storage medium,” can additionally refer to multiple structures, such a plurality of memories or computer-readable storage mediums. As referred to herein, a “memory” can comprise any type of computer-readable storage medium unless otherwise specified. A computer-readable storage medium can store instructions for execution by at least one processor, including instructions for causing the processor to perform steps or stages consistent with an embodiment herein. Additionally, one or more computer-readable storage mediums can be utilized in implementing a computer-implemented method. The term “computer-readable storage medium” should be understood to include tangible items and exclude carrier waves and transient signals.

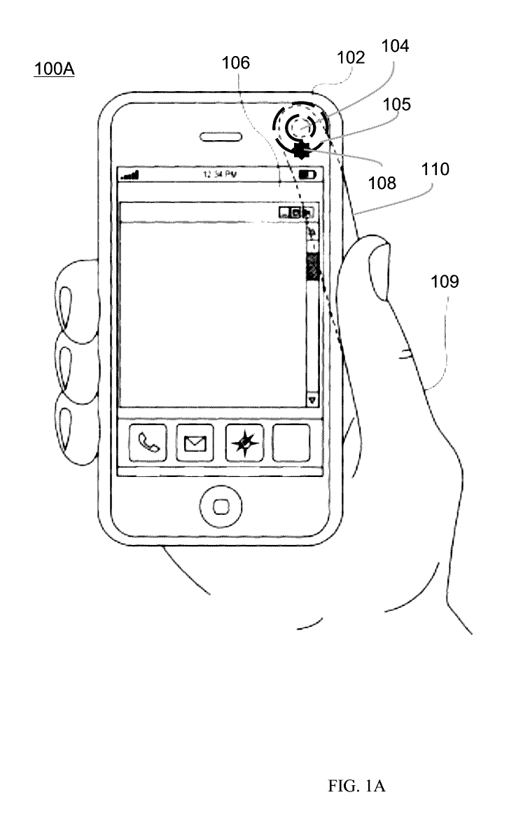

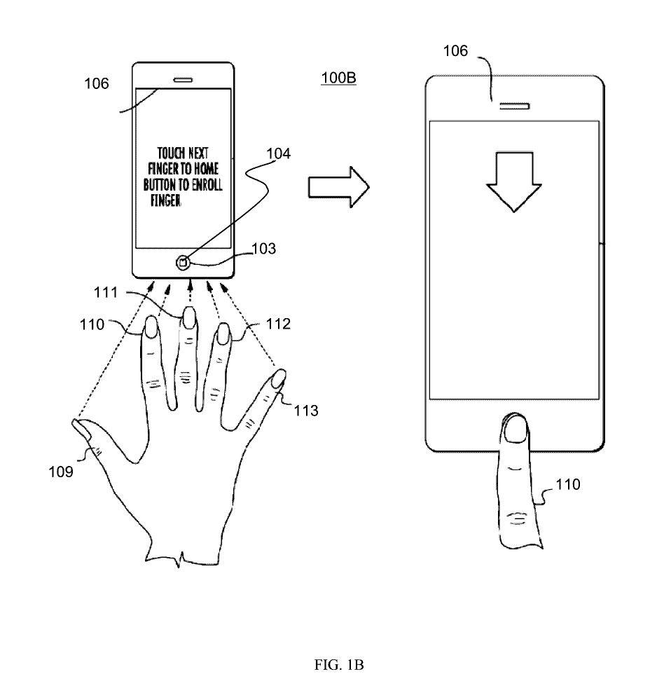

FIG. 1A shows an example of a user device 100A for non-invasive measurement of blood glucose content in which various implementations as described herein may be practiced. In the presently described embodiment, the user device 100A is a mobile device such as a smartphone. In other embodiments, however, the user device 100A could be other types of electronic devices, such as a wearable device that can be secured to a body member of the patient using a mount such as a wristband, strap, temples or similar types of supports.

As shown in FIG. 1A, the user device 100A includes a housing 102 that supports various components, including a detector 104 such as an infra-red (IR) camera, and a display 106 for presenting graphical user interfaces (GUIs) displaying information to a patient from apps running on the user device 100A, such as a health monitoring app or a browser application. In the presently described example, the detector 104 is a built-in unit. In other example embodiments, the detector 104 is not integrated within the housing 102, and detachably mounted on the housing 102 using a mount.

As shown in FIG. 1A, a user can place a vascularized appendage, such as an index finger 110, proximate to the detector 104. The detector 104 includes one or more sensors (not shown) for sensing the transmitted, absorbed or luminescence radiation from the glucose molecules contained in the blood circulating in a site. The site is a vascularized organ, part, or appendage 109 of the user's body. Accordingly, the site contains blood vessels or an arrangement of blood vessels that allow blood containing glucose to flow through the site.

Soluble molecules of glucose in the blood have a strong absorbance of NIR and mid-Infrared radiation that are proportional to the amount of glucose in the blood. With increasing concentration of glucose in blood, there is less radiation at specific wavelengths that is reflected back. Infra-red spectroscopy is known to be an effective method for measuring the reflected radiation non-invasively. The sensors of detector 104 are sensitive in the infra-red region of the blood glucose radiation, and therefore configured to measure, monitor, or report infra-red energy absorption of blood glucose in the characteristic infra-red absorption spectrum ranges. The sensors of detector 104 could be a camera or photodiodes or the like that are capable of detecting IR and NIR signals.

In some examples, each sensor in the detector 104 is coupled with one or more optical filters (not shown), such as narrow band filters or broadband filters to improve isolation or detection of the target infra-red radiation emanating by the blood glucose.

If the intensity of the radiation measured by the detector 104, and particularly the NIR, is weak, the signal from the detector 104 can be fed to one or more amplifiers (now shown) coupled to detector 104 so as to amplify the signal. However, amplification is usually not required for red, IR, and green light signals, as their attenuation does not pose a problem in measuring the glucose content of the blood found at the site. In the presently described examples, NIR and IP signals are chosen for measurement by the detector 104 since these signals can be detected at low intensities, thereby detectible by low sensitive, low cost detector 104.

In some example implementations such as the embodiment of FIG. 1A, user device 100A further includes additional detector(s) 105 having sensors to measure the user's location, altitude, acceleration, pulse, temperature, blood pressure, blood oxygen O2 or blood carbon dioxide CO2 levels, sweat, saliva, urine, tear, or other biometric information pertaining the user to provide improved resolution of blood glucose detection method herein. Output from the detector(s) 105 provide additional metrics for determining the blood glucose content and are analyzed and processed in conjunction with the measurements obtained from the infra-red sensors of detector 104. For example, measurements from detector 104 can be preferably synchronized with readings obtained from a pulse sensor of detector 105 to determine the systole and diastole of the user's cardiac cycle, so that the infra-red signal measurement at the veins and tissues may be adjusted based on the cardiac cycle. Similarly, it is known that as temperature rises, radiation energy is increased. Accordingly, body temperature readings obtained from a temperature sensor of the detector 105 can be utilized to improve interpretation of spectral distribution of the infra-red signals measured by detector 104 by compensating for the variations in the temperature of the vascularized appendage at the site being tested.

Likewise, pulse oximetry using a pulse oximeter probe can be used to measure blood oxygen. Pulse oximetry uses red and IR light to distinguish between hemoglobin and oxy-hemoglobin in the blood, on which further processing is applied to get the oxygen saturation.

In the event the radiation from the blood glucose does not have sufficient amplitude for accurate determination of the glucose content, the user device 100A can optionally include one or more radiation devices 108 such as photodiodes or lasers to irradiate the blood circulating in the site.

The components and arrangements shown in FIG. 1A are not intended to limit the disclosed embodiments, as the user device 100A components used to implement the disclosed processes and features can vary. The placement of the detector 104 and detector 105 can be optimized based on the application. For example, the housing 102 may include multiple parts, and be configured as a clamshell or a slider device in contrast to the candy bar design shown in FIG. 1A. Similarly, the user device 100A can be a wearable such as a smart watch with detector 104 placed, for example, on the back of the smart watch resting upon the patient's wrist and further including detector 105 in a strap or wristband. The user device 100A can also be a smart glasses, with detector 104 or detector 105 placed on the nose support, temples, or other parts of the smart glasses frame.

Moreover, the placement of the components of the user device 100A may vary depending on the implementation. For instance, the detector 104 can be integrated within the display 106, or positioned along on a side of the display 106. In such implementations, the detector 104 can measure the radiation pattern from blood glucose molecules when the user device 100A is placed proximate to a body member, for example when the user places the user device 100A against an ear while managing a phone call or a voicemail. It has been observed that the blood flow can be reasonably measured in earlobes due to the thinner tissue and lack of hard structures such as bones or cartilage in the earlobes.

When the user device 100A is not provisioned with detector 104 or detector 105, these devices can be provided as add-on that can be placed on the housing 102 or display 106 of the user device 100A. Accordingly, a kit may be provided including a user device 100A, as well as one or more detector 104 and/or detector 105 that are adapted to be communicatively coupled to the user device 100A to transmit signals relating to measured values to one or more processors of the user device 100A.

In the example embodiment of FIG. 1B, the detector 104 of user device 100B is positioned below the bottom side of display 106 to facilitate positioning the patient's index finger thereupon. As shown in FIG. 1B, the detector 104 is integrated with a physical button 103 that is also adapted to perform other functions corresponding to the operation of the user device 100B, such as a home button that upon activation launches the home screen GUI including home screen icons associated with various apps operable on the user device 100B. In the presently described embodiment, the user can enroll the finger 109-113 so that the glucose measurements are calibrated and calculated according to the size, shape, and/or characteristics of the enrolled finger 109-113. The patient can optionally register other fingers 109-113 for blood sugar measurement, and obtain glucose readings using the registered finger 109-113.

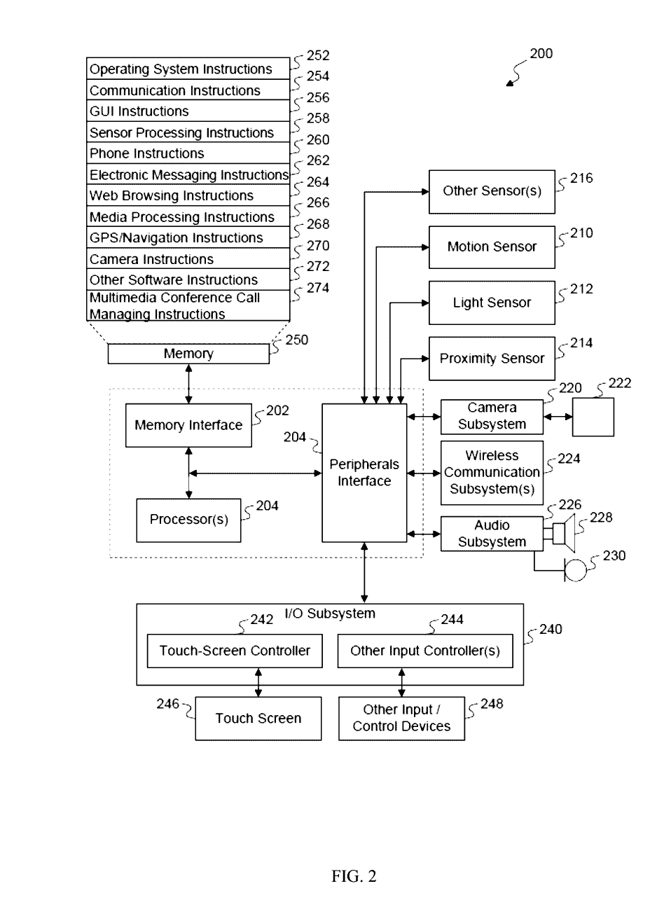

Reference is now made to FIG. 2, which shows a diagram of an example of a user device 200. The user device 200 can be used to implement computer programs, applications, methods, processes, or other software to perform embodiments described in the present disclosure, such as the user devices 100A of FIG. 1A or 100B of FIG. 1B. The user device 200 includes a memory interface 202, one or more processors 205 such as data processors, image processors and/or central processing units, and a peripherals interface 206. The memory interface 202, the one or more processors 205, and/or the peripherals interface 206 can be separate components or can be integrated in one or more integrated circuits. The various components in the user device 200 can be coupled by one or more communication buses or signal lines.

Sensors, devices, and subsystems can be coupled to the peripherals interface 206 to facilitate multiple functionalities. For example, a motion sensor 210, a light sensor 212, and a proximity sensor 214 can be coupled to the peripherals interface 206 to facilitate orientation, lighting, and proximity functions. Other sensors 216 can also be connected to the peripherals interface 206, such as a positioning system (e.g., GPS receiver), a temperature sensor, a biometric sensor, or other sensing devices, to facilitate related functionalities. A GPS receiver can be integrated with, or connected to, the user device 200. For example, a GPS receiver can be built into mobile telephones, such as smartphone devices. GPS software allows mobile telephones to use an internal or external GPS receiver (e.g., connecting via a serial port or Bluetooth®). A camera subsystem 220 and an optical sensor 222, e.g., a charged coupled device (“CCD”) or a complementary metal-oxide semiconductor (“CMOS”) optical sensor, and/or photodiodes, may be utilized to facilitate camera functions, such as recording photographs and video clips. In some example embodiments, the camera subsystem 220 includes a detector such as the detector 104 and/or detector 105 of FIG. 1A for measuring infra-red radiation from blood glucose. The camera subsystem 220 may optionally also include, in some example embodiments, a radiation source for irradiating a site. The radiation source may be adapted to irradiate the site at selected red, IR or NIR frequencies.

Communication functions may be facilitated through one or more wireless/wired communication subsystems 224, which includes a Ethernet port, radio frequency receivers and transmitters and/or optical (e.g., infra-red) receivers and transmitters. The specific design and implementation of the wireless/wired communication subsystem 224 depends on the communication network(s) over which the user device 200 is intended to operate. For example, in some embodiments, the user device 200 includes wireless/wired communication subsystems 224 designed to operate over a GSM network, a GPRS network, an EDGE network, a Wi-Fi or WiMax network, and a Bluetooth® network. Accordingly, the communication subsystems 224 can be configured to communicate biometric data such as blood glucose concentration over such networks or the like.

An audio subsystem 226 may be coupled to a speaker 228 and a microphone 230 to facilitate voice-enabled functions, such as voice recognition, voice replication, digital recording, and telephony functions.

The communication interface 240 includes a touch screen controller 242 and/or other input controller(s) 244. The touch screen controller 242 is coupled to a touch screen 246. The touch screen 246 and touch screen controller 242 can, for example, detect contact and movement or break thereof using any of a plurality of touch sensitivity technologies, including but not limited to capacitive, resistive, infra-red, and surface acoustic wave technologies, as well as other proximity sensor arrays or other elements for determining one or more points of contact with the touch screen 246. While a touch screen 246 is shown in FIG. 2, the communication interface 240 may include a display screen (e.g., CRT or LCD) in place of the touch screen 246. The touch screen 246 can, for example, also be used to implement virtual or soft buttons and/or a keyboard.

The other input controller(s) 244 is coupled to other input/control devices 248, such as one or more buttons, rocker switches, thumb-wheel, infra-red port, sensors, detectors (such as detectors 104 and/or detectors 105 of FIG. 1) USB port, and/or a pointer device such as a stylus. The input/control devices 248 are coupled to the communication interface 240 and can communicate with the other input controller(s) 244 by optical, wired, or wireless communications such as short range communications, Bluetooth®, near field communication (NFC), etc.

The memory interface 202 is coupled to memory 250. The memory 250 includes high-speed random access memory and/or non-volatile memory, such as one or more magnetic disk storage devices, one or more optical storage devices, and/or flash memory (e.g., NAND, NOR). The memory 250 stores an operating system 252, such as DRAWN, RTXC, LINUX, iOS, UNIX, OS X, WINDOWS, or an embedded operating system such as VXWorkS. The operating system 252 can include instructions for handling basic system services and for performing hardware dependent tasks. In some implementations, the operating system 252 can be a kernel (e.g., UNIX kernel).

The memory 250 may also store communication instructions 254 to facilitate communicating with one or more additional devices, one or more computers and/or one or more servers. The memory 250 can include GUI instructions 256 to facilitate GUI processing; sensor processing instructions 258 to facilitate sensor-related processing and functions; phone instructions 260 to facilitate phone-related processes and functions; electronic messaging instructions 262 to facilitate electronic-messaging related processes and functions; web browsing instructions 264 to facilitate web browsing-related processes and functions; media processing instructions 266 to facilitate media processing-related processes and functions; GPS/navigation instructions 268 to facilitate GPS and navigation-related processes and instructions; camera instructions 270 to facilitate camera-related processes and functions; and/or other software instructions 272 to facilitate other processes and functions. The memory 250 may also include multimedia conference call managing instructions 274 to facilitate conference call related processes and instructions.

In some embodiments, the communication instructions 254 may include software applications to facilitate connection with a server that manages communications between the user device 200 and other devices such as medical equipment (for example laboratory diagnosis devices), and the GUI instructions 256 may include a software program that facilitates a patient associated with the user device 200 to receive messages from the server, provide user input, and so on. Further, the communication instructions 254 may include software applications for a patient associated with the user device 200 to transmit, over a communication channels that may be secured or unsecured, such as the Internet, data including biometric data such as patient's blood glucose level, blood pressure, oxygen level, etc. to the server. The GUI instructions 256 may include software program that facilitates a patient associated with the user device 200 to select a portion of biometric data for providing to medical equipment in communication with the server, send and/or receive messages from the server relating to biometric data for further diagnosis or analysis by the medical equipment, receive instructions concerning the biometric data or calibration of sensor(s) or detector(s), and so on.

Each of the above identified instructions and applications may correspond to a set of instructions for performing one or more functions described above. These instructions need not be implemented as separate software programs, procedures, or modules. The memory 250 may include additional instructions or fewer instructions. Furthermore, various functions of the user device 200 may be implemented in hardware and/or in software, including in one or more signal processing and/or application specific integrated circuits.

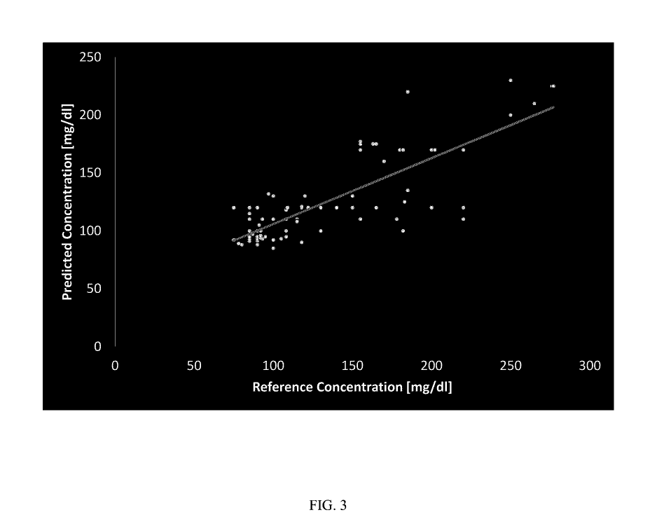

FIG. 3 is a graph depicting the reference and predicted correlation of blood glucose concentration. Measure the level of glucose in blood accurately depends on the transmittance of NIR and IR radiation that are related to the amount of blood in the path of the lights. In other words, for the same glucose level, a large amount of blood will result in lower transmittance, whereas less blood will result in a larger transmittance. The glucose value needs to be scaled according to the amount of blood residing inside the site at a time of measurement. In some examples, the amount of blood can be estimated by measuring the blood oxygen levels using an oxygen O2 sensor in detector 105. As explained in FIG. 3, predicted concentration of blood sugar level over reference level using NIR detection only is shown. As indicated in FIG. 3, detection of the NIR radiation generally provides suitable results for measuring the blood sugar content. Improvements in the calculation of the blood sugar level can be achieved by also measuring the red and IR radiations. In other examples, further improvements in the accuracy are possible by measuring the red, IR and NIR patterns, as well as the blood oxygen level.

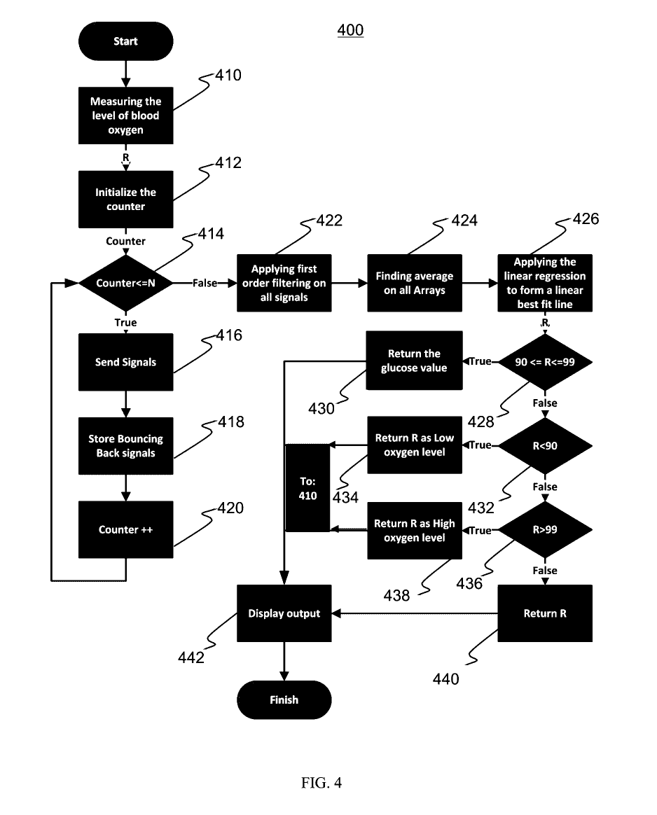

FIG. 4 is a flow chart showing a method for measuring blood glucose content in accordance with the present disclosure. As mentioned earlier, in order to measure the blood glucose accurately, there is a need to determine whether the blood in the site is at the required volume. The blood oxygen saturation levels can be determined non-invasively using near-infrared spectroscopy sensed by detector 104 and/or detector 105. Accordingly, Algorithm-1 outlined below is used for measuring blood oxygen. This algorithm utilizes pulse oximetry procedure to measure the oxygen saturation within the peripheral circulation while verifying that the circulation is sufficient for measurement of glucose concentration at the site. Pulse oximetry procedure is considered to be a non-invasive, painless, general indicator of oxygen delivery to the peripheral tissues (such as the finger, earlobe, or nose) of the site. This procedure utilizes different characteristics of blood cells to determine the oxygen level in the body. The blood cells can absorb the specific lights such as red and infrared lights. There is a color difference between blood cells saturated with oxygen and blood cells without oxygen. The saturated blood cells are red while the other ones are darker which results in different light absorbance. The infrared light is absorbed more by the oxygen-rich blood cells while the red light is absorbed more by the blood cells without oxygen. Therefore, two different wavelength (red and IR) will be used to measure the changing of absorbance at each of the wavelength by detector 104 and/or detector 105. The AC components of both are filtered out from the raw signals by using a high pass filter while the DC components are computed by its low pass counterpart.

Thereafter, the oxygen level is scaled from 0-100 to determine the percentage of oxygen saturation. It is important to consider any medical conditions that may prevent blood flow to the site, which may result in accuracies in the blood oxygen level determination as explained further below.

Algorithm-1 pseudo code implementation of measuring blood oxygen

Input: red light signal
Output: level of blood oxygen
Begin Program
AC_{red} ← AC for red signal
DC_{red} ← DC for red signal
AC_{IR} ← AC for IR signal
DC_{red} ← DC for IR signal
R ← Blood Oxygen level
Send Red light
AC_{red} and DC_{red} := measure current for AC and DC of red signal
Send IR light
AC_{IR} and DC_{IR} := measure current for AC and DC of red signal
R := ({AC_{red}/DC_{red}}/{AC_{IR}/AC_{IR}}) * 100
End Program

After finding the oxygen level in the peripheral circulation, the next step is to calculate the amount of infra-red bouncing back signals based on different wavelength. It is observed that the higher the glucose concentration in the blood, the lower the infra-red signal at specific wavelengths is reflected back. As a result, the blood glucose level can be non-invasively determined by observing the amount of infra-red radiation reflecting at the site. According to the method proposed herein, three different wavelengths for NIR plus a separate wavelength for IR are used to measure the bouncing back signals from the blood site.

The NIR, IR and/or red light reflected by the glucose contained in the blood may be detectable by the detector 104 and/or detector 105. Otherwise, the radiation devices 108 generate these different wavelengths and measures the wavelengths that are reflected back. For each wavelength, sampling of N times will be considered. A single first order filter for each wavelength will be applied to reduce noise level while bringing the amplitudes of the different wavelengths onto the same level. Therefore, it will be possible to apply the same processing on all the wavelengths at the same time. Thereafter, the average of all variables such as fat tissue, skin thickness and bone presence or density will be calculated for different wavelength to measure the wavelengths reflected back. Details of the algorithm are introduced in Algorithm-2.

Algorithm-2 pseudo code implementation of measuring
bouncing back NIR and IR signals

```
Input: NIR and IR signals with different wavelength
Output: Average bouncing back NIR and IR signals
Begin Program
NIRₗ[0 ... N] ← array of low wavelength NIR Light
NIRₘ[0 ... N] ← array of medium wavelength NIR Light
NIRₕ[0 ... N] ←array of high wavelength NIR Light
IR[0 ... N] ← array of IR Light
Loop from i = 1 to N
    Send low wavelength NIR light
    NIRₗ[i] := bouncing back NIR signal
    Send medium wavelength NIR light
    NIRₘ[i] := bouncing back NIR signal
    Send high wavelength NIR light
    NIRₕ[i] := bouncing back NIR signal
    Send IR light
    IR[i] := bouncing back IR signal
End loop
Loop from i = 1 to N
Applying first order filtering on NIRₗ[i], NIRₘ[i], NIRₕ[i] and IR[i]
End loop
// finding the average
NIRₗ := Σᵢ₌₁^{N} NIRₗ[i]/N
NIRₘ := Σᵢ₌₁^{N} NIRₘ[i]/N
NIRₕ := Σᵢ₌₁^{N} NIRₕ[i]/N
IR := Σᵢ₌₁^{N} IR[i]/N
Return NIRₗ, NIRₘ, NIRₕ, and IR
End Program
```

Lastly, the glucose levels are computed. To present the range of the results from the Algorithm-2 (filtered-average samples), the minimum value is considered as the beginning of the scale (minᵥₐₗᵤₑ), and the maximum value of samples as the endpoint of the scale (maxᵥₐₗᵤₑ). Therefore, the range of the samples is between [(minᵥₐₗᵤₑ), (maxᵥₐₗᵤₑ)]. These samples resulted from Algorithm-2 are interpolated to form a linear best fit line through known linear regression techniques. Central value of this line (C) represents the glucose value. It is required to map the glucose value into appropriate scale to show low, normal, or high blood sugar. Usually the scale for the glucose value is [55,355] mg/dl. A simple normalization is applied to map the glucose value to the appropriate range as follows: 55+(C−minᵥₐₗᵤₑ)×[(355−55)/(maxᵥₐₗᵤₑ−minᵥₐₗᵤₑ)].

It is noted that the glucose level accuracy can be improved by considering the blood oxygen level. In some example embodiments, pre-exiting or known physical or immunological conditions of the user such as lung diseases or high or low blood pressures are factored in the blood oxygen calculations. For example, if it is known that the user suffers from lung disease and as a result has a lung capacity less than normal causing the user's blood oxygen level to drop by a multiple (e.g. 20% less), then the multiple is factored in and the blood oxygen calculations are scaled to avoid discrepancies in the determination of the overall blood sugar level.

In the events of low or very high oxygen saturations, the results may become unacceptable, and must be normalized taking into consideration the excess or lack of oxygen in order to achieve meaningful results. The algorithm-3 presents the details of the final steps to measure the glucose level.

Algorithm-3 pseudo code implementation of measuring
glucose by using NIR and IR signals

```
Input: average NIR and IR signals with different wavelength
Output: the blood glucose level
Begin Program
Applying the linear regression to form a linear best fit line
Line: aNIRₗ + b NIRₘ + cNIRₕ + IR + e = 0
C := the cental value of line
IF 90 ≦ R ≦ 99 THEN
Return 55 + (C − minᵥₐₗᵤₑ) × [(355 − 55)/ (maxᵥₐₗᵤₑ − minᵥₐₗᵤₑ)] // Normal
Oxygen level
ELSE
    IF R < 90 THEN
        Return R as low oxygen value
    IF R > 99 THEN
        Return R as high oxygen value
End Program
```

Referring again to FIG. 4, there is shown a flow chart outlining the steps for implementing an example method for measuring the blood glucose level in the site. The steps associated with this example method may be performed by, for example, the user devices 100A or 100B of FIG. 1A or 1B, respectively, by one or more processors 205.

At step 410, upon placement of a user's site having a peripheral circulation proximate to a sensor such as the detector 105 of the user device 100A, the level of blood oxygen in the site is determined (i.e. measured or detected) by the detector (i.e. R value in FIG. 4) and communicated to the processor of the user device. At step 412, the processor initializes a counter N. Thereafter, at step 414, the processor compares the current count to N. If the current count is less or equal to N (i.e. True), then the processor at step 416 sends a signal to instruct a radiation source, such as the radiation devices 108 of FIG. 1A, to irradiate the site for a time period that may relate to the duration of the blood glucose level determination. The radiation source is configured to irradiate the site at selected red, IR or NIR frequencies.

At step 418, the detector measures the amount of radiation bouncing back from the blood, and sends this data to the processor, that stores medium wavelength NIR and high wavelength NIR radiation patterns detected at steps 414-418 values in arrays. At step 420, the processor increments the counter and repeats steps 414 to 418 so long as the condition at step 414 remains valid.

If the condition at step 414 becomes invalid (i.e. False), namely that the counter is larger than N, then at step 422, the processor applies a first order filter to low wavelength NIR, medium wavelength NIR and high wavelength NIR radiation patterns stored in arrays. Accordingly, the values in the arrays are normalized at step 422 by applying first order filtering. A first order filter is a filter that passes signals with a frequency lower than a certain cutoff frequency and attenuates signals with frequencies higher than the cutoff frequency. As a result, all stored values will be in the same normalized range. This is a procedure to eliminate irregularity (very high or very low) in the calculated values.

A processor of the user device then, at step 424, determines the average of all the arrays, then at step 426 performs a linear regression on the array values to form a linear best fit line. Central value of this line represents the glucose value. It is calculated to determine the actual blood glucose level at step 430. The glucose level accuracy is based on blood oxygen level. At step 428, the blood oxygen level (i.e. the R value in FIG. 4) is checked to determine whether it is in the normal range of 90 to 99 percent. If the blood oxygen level is in this normal range, the central value of the line (i.e. referred to as C in FIG. 4) is the glucose level. This value is mapped to [55,355] mg/ml to present the low, normal or high blood sugar by using simple normalization at step 442. Afterwards, the scaled value is provided to a communication interface (for example the communication interface 240 of FIG. 2) at step 442 for display on a display of the user device such as display 106 of user device 100A of FIG. 1A. The glucose level denotes the concentration of the glucose in the user's blood as detected at the site.

Otherwise, the blood oxygen level of less than 90 at step 432 is presented as low oxygen value at step 434, and the blood oxygen level of more than 99 at step 436 is presented as high oxygen value at step 438. In the situation of low or very high oxygen saturation, the result is not acceptable and the process as to jump back to step 410 to re-assess the level of blood oxygen. However, if the blood oxygen level is within the normal range, optionally at step 440 said oxygen level is provided to the communication interface, to be optionally at step 442 displayed on a display of the user device.

In the preceding disclosure, various example embodiments have been described with reference to the accompanying drawings. It will, however, be evident to those skilled in the art that modifications or changes may be made thereto, and additional embodiments may be implemented, without departing from the broader scope of the disclosure as set forth in the claims that follow and their equivalents. For example, some of the steps of the method can be performed by a server in communication with the user device, or by endpoint devices coupled to the server or the user device. The disclosure and drawings are accordingly to be regarded in an illustrative rather than restrictive sense.

What is claimed is:

1. A user device for non-invasive measurement of concentration of glucose contained in the blood in a site having a vasculature, comprising:
a display;
a first detector configured to detect an infra-red (IR) signal reflected from glucose contained in the blood at the site;
a communication interface coupled to the first detector;
at least one processor in communication with the communication interface and configured to:
detect the concentration of glucose contained in the blood at the site based on IR signal detected by the first detector; and
display a number representative of the concentration on the display.

2. The user device of claim 1, further comprising a second detector in communication with the communication interface and configured to detect the concentration of oxygen contained in the blood at the site.

3. The user device of claim 2, wherein the processor is further configured to:
detect the concentration of oxygen in the blood at the site detected by the second detector.

4. The user device of claim 2, wherein the first detector is further configured to detect red, and near infra-red (NIR) radiation.

5. The user device of claim 3, wherein the detecting of the concentration of oxygen in the blood at the site is by pulse oximetry technique.

6. The user device of claim 1, further comprising a radiation source irradiating the site.

7. The user device of claim 1, wherein the first detector is configured to be mounted on an external body surface proximate to a portion of the site.

8. The user device of claim 2, wherein the second detector is configured to be mounted on an external body surface proximate to a portion of the site.

9. The user device of claim 1, wherein the first detector is mounted using a wristband or a strap.

10. The user device of claim 2, wherein the second detector is mounted using a wristband or a strap.

11. A method for non-invasive measurement of concentration of glucose contained in the blood in a site having a vasculature, comprising:
detecting the concentration of glucose contained in the blood at the site based on infra-red (IR) signal; and
displaying a number representative of the concentration on a display.

12. The method of claim 11, wherein the step of detecting the concentration of glucose contained in the blood at the site includes measuring the red and near infra-red (NIR) radiation from the site.

13. The method of claim 12, further comprising the step of normalizing the measured red, infra-red (IR) and near infra-red (NIR) radiation by applying a first order filter to low wavelength NIR, medium wavelength NIR and high wavelength NIR radiation.

14. The method of claim 13, further comprising the step of applying linear regression to normalized measured red, infra-red (IR) and near infra-red (NIR) radiation.

15. The method of claim 14, further comprising the step of detecting the concentration of oxygen contained in the blood at the site.

16. The method of claim 15, further comprising determining whether the concentration of oxygen contained in the blood at the site is within a range of 90 to 99 percent.

17. The method of claim 16, responsive to the concentration of oxygen contained in the blood at the site being within a range of 90 to 99 percent, mapping a concentration of glucose contained in the blood at the site to [55,355] mg/ml.

18. The method of claim 17, further comprising the step of scaling the concentration of glucose contained in the blood at the site by a factor based on pre-existing condition of the user.

19. A kit for non-invasive measurement of concentration of glucose contained in the blood in a site having a vasculature, the kit comprising:
a first detector configured to detect red, infra-red, and near infra-red signals reflected from glucose contained in blood;
a user device including:
a communication interface coupled to the first detector;
at least one processor in communication with the communication interface and configured to:
detect the concentration of glucose contained in the blood at the site based on IR signal detected by the first detector; and
display a number representative of the concentration on the display.

20. The kit of claim 19, further comprising a second detector in communication with the communication interface and configured to detect the oxygen concentration contained in blood.
